SEQUENCE LISTING (2 sequences)

SEQ ID NO: 1 - protein, 645 aa, Setaria digitata
MSKNAIGIDLGTTYSCVGVFMHGKVEIIANDQGNRTTPSYVAFTDTERLIGDAAKNQVAMNPHNTVFDAKRLIGRKFDDGSVQSDMKHWPFKVMNAGGGKPKVQVEYKGETKTFTPGEISSMVLVKMKETAEAFLGHAVKDAVITVPAYFNDSQRQATKDSGAIAGLNVLRIINEPTAAAIAYGLDKKGHGERNVLIFDLGGGTFDVSILTIEDGIFEVKSTAGDTHLGGEDFDNRMVNHFVAEFKRKHKKDLASNPRALRRLRTACERAKRTLSSSSQASIEIDSLFEGIDFYTNITRARFEELCADLFRSTMDPVEKALRDAKMDKAQVHDIVLVGGSTRIPKVQKLLSDFFSGKELNKSINPDEAVAYGAAVQAAILSGDKSEAVQDLLFVDVAPLSLGIETAGGVMTALIKRNTTIPTKTSQTFTTYSDNQPGVLIQVYEGERAMTKDNNLLGKFELSGIPPAPRGVPQIEVTFDIDANGILNVSAQDKSTGKQNKITITNDKGRLSKDEIERMVQEAEKYKADDEAQKDRIAAKNALESYAFNMKQTIEDEKLRDKLSEEDKKKIQEKCDETVRWLDGNQTAEKDEFEHRQKELEAVSNPIITKLYQSAGGMPGGMPGGMPGGAPGGGSGGSGPTIEEVD

SEQ ID NO: 2 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

(19) United States
(12) Patent Application Publication
(10) Pub. No.: US 2023/0105103 A1
(43) Pub. Date: Apr. 6, 2023

(54) THERAPEUTIC MOLECULES FOR COMBATING SEPSIS

(71) Applicant: CENTRE FOR CELLULAR AND MOLECULAR PLATFORMS, (US)

(72) Inventors: Taslimarif SAIYED, Bangaluru (IN); Paresh Brijalal PATEL, Vadodara (IN); Balachandran RAVINDRAN, Bhubaneswar (IN); Santosh PANDA, Bhubaneswar (IN)

(73) Assignee: CENTRE FOR CELLULAR AND MOLECULAR PLATFORMS, Bangaluru (IN)

(21) Appl. No.: 17/789,381

(22) PCT Filed: Dec. 26, 2020
(86) PCT No.: PCT/IB2020/062459

§ 371 Date: Jun. 27, 2022

(30) Foreign Application Priority Data
Dec. 26, 2019 (IN) 201941053971

(51) Int. Cl. A61K 38/17; A61K 31/702; A61P 37/02

(52) U.S. Cl. CPC A61K 38/1767; A61K 31/702; A61P 37/02

(57) ABSTRACT

The present invention provides therapeutic molecules for combating sepsis. The invention provides proteins capable of improving survival through immunomodulation in post septicaemia in mammals and oligosaccharides, capable of immuno modulating inflammation in a mammal. The invention provides protein homologs of human HSP70 derived from nematode Setaria digitata and their recombinant forms and oligosaccharides and their role in immunomodulating inflammatory response in a mammal. The invention also provides a composition comprising therapeutic and/or prophylactic proteins and oligosaccharide molecules for immunomodulation in sepsis; and method for treating and/or preventing sepsis, MODS (multiple organ dysfunction syndrome) or septic shock in a mammal.

### FIELD OF THE INVENTION

The present invention relates to therapeutic and/or prophylactic molecules for combating sepsis and proteins capable of improving survival through immunomodulation in post septicaemia in mammals. The proteins and oligosaccharides are capable of modulating inflammation in mammals. The present invention also relates to protein homologs of human HSP70 derived from nematode Setaria digitata and their recombinant forms and oligosaccharides and their role in immuno-moculating inflammatory response in a mammal. The invention also provides a composition comprising therapeutic and/or prophylactic proteins and oligosaccharide molecules for immunomodulation in sepsis; and method for treating and/or preventing sepsis. MODS (multiple organ dysfunction syndrome) or septic shock in a mammal.

### BACKGROUND OF THE INVENTION

Sepsis is a systemic inflammatory response mediated by various innate immune cells, including neutrophils, monocytes and macrophages, upon severe infection (Stearns-Kurosawa et. al., 2011). Normally, the moderate production of pro-inflammatory cytokines, including tumor necrosis factor (TNF-α), interleukins (IL-1, IL-6 and IL-8) assist in confining infection and restricting tissue damage; with the clearance of the infectious agent, the inflammatory response can recover to homeostasis. However, excessive and prolonged production of inflammatory cytokines can lead to an overwhelming inflammatory response, which is referred to as sepsis (Cai et. al., 2010). Thus, Sepsis is a life-threatening infection which impairs survival outcomes for patients as sepsis requires swift diagnosis and treatment to lower the risk of mortality. A ‘golden hour’ is believed to exist, in which treatment is vital to lower the risk of death. Antimicrobial resistance is a major factor determining clinical unresponsivencss to treatment and rapid evolution to sepsis and septic shock. Sepsis patients with resistant pathogens have been found to have a higher risk of hospital mortality.

The mortality rate in severe sepsis can reach as high as 70% and the number of cases of sepsis continues to increase due to the rising number of immunocompromised patients (Russell, 2006; Matsuda et. al., 2012). The molecular mechanism of sepsis remains to be fully elucidated. Studies have revealed that several mechanisms may contribute to the occurrence of sepsis, including the continued activation of neutrophils and macrophages/monocytes, upregulation of lymphocyte costimulatory molecules (Nolan et. al., 2008; Flohé et. al., 2006), rapid lymphocyte apoptosis and delayed neutrophil apoptosis, and excessive necrosis of cells and tissues (Roger et. al., 2012; Paunel-Görgülü et. al., 2012).

In sepsis, gram-negative bacterial infection was found to be predominant and their endotoxin such as lipopolysaccharide (LPS) is sensed by immune receptors such as Toll like receptor (TLR4). The previous approaches for treating sepsis were limited to breaking interaction between LPS and TLR4 by using chemical antagonist. TLR41 has been explored widely as a potential target in sepsis and various molecules, which antagonize PAMPs and restrict the TLR41 activation, have been tested for sepsis intervention (Wittebole et al., 2010). However, some of these LPS antagonists were not found to be useful in later clinical trials (Opal, 2013). Studies have indicated that TLR2 plays a role in polymicrobial sepsis by regulating neutrophil migration (Alves-Filho et al., 2009). Mycobacterium indicus pranii activates the TLR2 mediated MyD88 signaling pathway resulting higher activation of NF-kB/AP-1 (Kumar et al., 2014). Eritoran is one such example that could bind to TLR4 and compete with LPS. It was only antagonist in nature and its binding to TLR4 did not initiate any signaling. There are many other such molecules that have been tried for sepsis intervention. Majority of previous interventions were found to be effective only if injected at very early time points after sepsis onset while later interventions were not effective. Recent clinical studies with Mycobacterium indicus pranii, have shown improvement in clinical outcomes in sepsis patients (Sehgal et al., 2015). Immunomodulation through TLR2 to restore immunological equilibrium as a therapeutic strategy for sepsis and use of different natural TLR2 ligands is less explored.

WO1993017712A2 relates to a conjugate compound comprising at least one heat shock protein or portion thereof including at least one immune-stimulatory domain and at least one capsular oligosaccharide or polysaccharide of pathogenic bacteria. The compound comprises oligosaccharides of Meningoccoi C (MenC) group and a heat shock protein selected from M.Bovis BCG GroEI type 65 kDa hsp (hspR65), recombinant M. tuberculosis DNaK-type 70 kDa hsp (hspR70) and a heat shock protein from H.pyroli.

Vinokurov et al., (2012) demonstrates that human recombinant HSP70 expressed in armyworm (Spodoptera frugiperda) ameliorates systematic inflammation including ROS and TNF-α in different myeloid cells after macrophage activation. It establishes exogenous Hsp70 as a promising pharmacological agent for the prophylactic treatment of various types of sepsis.

Kustanova et al., (2006) demonstrates that administration of exogeneous Hsp70 before and after LPS challenges can reduce mortality rates and modify several parameters of hemostasis and hemodynamics. Hsp70 isolated from bovine muscles showed significant protective effects against the impaired coagulation and fibrinolytic systems caused by LPS, and reduced the mortality caused by Escherichia coil and Salmonella typhimurium LPS injections significantly.

Motta et al., (2007) compared effects of LPS-free Mycobacterial tuberculosis hsp 70 (TBhsp70) and its possible contaminants on dendritic cells (DC). The results of the document show that TBhsp70 inhibits DC differentiation from bone marrow precursors, inducing IL-10 but not TNF-α thereby supporting the hypothesis that TBhsp70 does not have inflammatory potential, but rather has immunosuppressive properties.

US20100047272A1 discloses use of a mycobacterial heat-shock protein for manufacture of a vaccine for therapeutic application, more particularly a vaccine for treating animals infected with Mycobacterium comprising an immunologically effective amount of mycobacterial Hsp70 protein.

Chitin is one of the most abundant polymers present in nature and its deacetylated version, chitosan has been used for different applications (Casadidio et al., 2019). Chito-oligomers are composed of homo- or hetero-oligomers of N-acetylglucosamine and D-glucosamine with variable solubility in water.

Panda et al., (2012) disclose that chitohexaose activates macrophages by alternate pathway through TLR4 and blocks endotoxemia. Murine macrophages and human monocytes up regulated Arginase-1 and released high levels of IL-10 when incubated with chitohexaose. Macrophages of C3H/HeJ mice (non-responsive to LPS) failed to get activated by chitohexaose suggesting that a functional TLR4 is critical for alternate activation of macrophages also. Chitohexaose inhibited LPS induced production of inflammatory molecules TNF-α, IL-1b and IL-6 by macropahges in vitro and in vivo in mice. Intraperitoneal (IP) injection of chitohexaose completely protected mice against endotoxemia when challenged with a lethal dose of LPS. Furthermore, chitohexaose was found to reverse LPS induced endotoxemia in mice even 6/24/48 hours after its onset.

Fuchs et al., (2018) demonstrates that fungal ligand chitin directly binds TLR2 and triggers inflammation dependent on oligomer size. The document demonstrates that Chitin-TLR2 binding appears to involve free ends of at least 5 NAG long oligomers, which explains why fungal cells, where chitin is polymeric and cross-linked to other cell wall components, only have limited numbers of accessible TLR2 epitopes. The document further provides that TLR2 has a preference for free ends of ≥6 NAG, both in context of fungal cell wall and for soluble chitin oligomers.

Zhao et al., (2020) shows that chitoheptaose promotes heart rehabilitation in a rat myocarditis model by improving antioxidant, anti-Inflammatory, and antiapoptotic properties. The document demonstrates that chitoheptaose showed significant therapeutic effects and inflammatory properties by reducing serum levels of IL-1β. Among all chitosan oligosaccharides (COS), the COS>degrees of polymerization (DP) 4 showed anti-inflammatory activities (the activity order was chitopentaose<chitohexaose<chitoheptaose) by reducing levels of interleukin- (IL-) 1β, IL-17A, and interferon- (IFN-) γ and increasing the level of IL-10.

AU 2016200853 relates to anti-bacterial applications of poly-N-acetylglucosamine nanofibers and a method for treating a disease or a condition associated with a bacterial imbalance in a subject in need thereof, comprising topically administering a composition comprising shortened fibers of poly-β-1---4-N-acetylglucosamine (“sNAG nanofibers”) to a subject, wherein the sNAG nanofibers are less than 10 μm in length, wherein the sNAG nanofibers comprise 70% or more than 70% of N-acetylglucosamine monosaccharides, and wherein the sNAG nanofibers do not have an effect, or substantially have no effect, on bacterial growth or survival of Staphylococcus aureus bacterial cultures in vitro.

Okawa et al., (2003) demonstrates a comparative study of protective effects of chitin, chitosan, and N-acetyl chitohexaose (NACOS-6) against mice infected intravenously or intraperitoneally with Pseudomonas aeruginosa and Listeria monocytogenes. Mice pretreated with chitin, chitosan and NACOS-6 showed resistance to intraperitoneal infections by both microbes. Only mice pretreated with chitin and chitosan showed resistance to intravenous infections by both microbes. Suzuki et al., (1984) indicates that both chitin and chitosan are able to exhibit immunopotentiating action for lethal challenge of C. albicans. Mice treated with chitin showed a longer life span against challenge of C. albicans cells via the IV route, and chitosan-treated mice were more resistant than chitin-treated mice when the challenge was conducted via IP route.

Solov'eva. et al., (2013) demonstrates systematic studies of chitosan binding, with LPS and the biological properties of the chitosan-LPS complexes. Chitosan has been shown to interact specifically with LPS to form water-soluble stable complexes of various stoichiometry compositions. Chitosan, oligochitosan and the N-acylated derivatives were shown to protect against LPS-induced mortality in a D-galactosamine-sensitized mouse model.

EP14359761B1 discloses use of water-soluble chito-oligamers of N-acetyl glucosamine (NAG) and glucosamine for manufacture of a medicament for treatment of disorders such as joint disorders including osteoarthritis and rheumatoid arthritis and inflammatory disorder, wherein the chain length of the chile-oligomers is in the range of about 2-50, and wherein the degree of deacetylation is in the range of about 0-70%. However, the document does not disclose chito-oligomers for treatment or prophylaxis of sepsis and does not disclose or suggests specific length of chito-oligomer being capael of immune-modulating sepsis in a mammal.

In the field of art chitin and chito-oligomers are known for treating endotoxemia, sepsis and septic shock. The deacetylated or partially acetylated child-oligomers possess different anti-oxidant and anti-inflammatory activities based on mitogen-activated protein kinase (MAPK) signaling (Hyung et al., 2016). Prophylactic treatment of chitosan oligosaccharides was found to attenuate inflammation and oxidative stress and protect mice from LPS challenge (Qiao et al., 2011). TLR2 was identified by Fuchs et al. 2018 as a receptor for chitin and chito-oligomers of varying chain length, where chain length of 6 or more was found to be important for TLR2 based immune activation (Fuchs et al., 2018). However, the therapeutic effects of acetylated chito-oligomers on sepsis and specifically, which oligosaccharide of chito-oligomers plays a more important functional role still remain unclear.

Therefore, there is a need of potent sepsis inhibitor which acts faster to save the patients. Further, there is a need for a therapeutic agent and a prophylactic agent for immune-modulating sepsis in a subject in need thereof. Further. There is a need for pharmaceutical composition for medicinal, prophylactic, therapeutic, treatment of a subject suffering from sepsis.

The present invention provides protein homolog of human HSP70 derived from nematode Setaria digitata and a chito-oligosaccharide selected from a group comprising of a derivative of hexa-N-acetyl chitohexaose, hepta-N-acetyl chitoheptaose or octa-N-acetyl chitooctaose which is capable of treating and/or preventing sepsis, multiple organ dysfunction syndrome (MOOS) or septic shock. The protein is capable of immunomodulating the survival through post-septicaemia in a mammal. The invention further provides use of acetylated form of chito-oligomers, derivative thereof individually or in composition for immunomodulation. The invention also provides a method of treatment or prevention of sepsis, MODS or septic shock in a mammal including human comprising administering an effective amount of protein homolog of human HSP70 derived from nematode Setaria digitata or chito-oligosaccharides wherein the chito-oligosaccharides is selected from the group comprising of a derivative of hexa-N-acetyl chitohexaose, hepta-N-acetyl chitoheptaose or octa-N-acetyl chitooctaose or a combination thereof together with a pharmaceutically acceptable carrier thereof to said mammal.

### REFERENCES

1. Maxim Vinokurov, Vladimir Ostrov, Marina Yurinskaya, David Garbuz, Arkady Murashev, Olga Antonova, Mikhail Evgen'ev. Cell Stress Chaperones. 2012 Jan;17(1):89-101. Recombinant human Hsp70 protects against lipoteichoic acid-induced inflammation manifestations at the cellular and organismal levels. doi: 10.1007/s12192-011-0288-0.

2. Gul'sara A Kustanova, Arcady N Murashev, Vadim L Karpov, Boris A Margulis, Irina V Guzhova, Izabella R Prokhorenko, Sergei V Grachev, Michael B Evgen'ev, Cell Stress Chaperones. Autumn 2006; 11(3):276-86. Exogenous heat shock protein 70 mediates sepsis manifestations and decreases the mortality rate in rats. doi: 10.1379/cse-195r.1.

3. Alves-Filho, J. C., Freitas, A., Souto, F. O., Spiller, F., Paula-Neto, H., Silva, J. S., Gazzinelli., R. T., Teixeira, M. M., Ferreira, S. H., & Cunha, F. Q. (2009). Regulation of chemokine receptor by Toll-like receptor 2 is critical to neutrophil migration and resistance to polymicrobial sepsis. Proceedings of the National Academy of Sciences of the United States of America, 106(10), 4018-4023. https://doi.org/10.1073/pnas.0900196106.

4. Adriana Motta, Carla Schmitz Luiz Rodrigues Flávia Ribeiro Cesar Teixeira Thiago Detanico Carla Bonan Heather Zwickey Cristina Bonorino. September 2007 Immunology 121(4):462-72. Mycobacterium tuberculosis heat-shock protein 70 impairs maturation of dendritic cells from bone marrow precursors, induces interleukin-10 production and inhibits T-cell proliferation in vitro. DOI 10.1111/j.1365-2567.2007.02564.x.

5. Yoshio Okawa, Makiko Kobayashi, Shigeo Suzuki, Masuko Suzuki. Biol. Pharm Bull. 2003 Jun: 26(6):902-4. Comparative study of protective effects of chitin, chitosan, and N-acetyl chitohexaose against Pseudomonas aeruginosa and Listeria monocytogenes infections in mice. doi: 10.1248/bpb.26.902.

6. K Suzuki, Y Okawa, K Hashimoto, S Suzuki, M Suzuki. Microbiol Immunol 1984;28(8):903-12. Protecting effect of chitin and chitosan on experimentally induced murine candidiasis. doi: 10.1111/j.1348-0421.1984.tb00746.x.

7. Tamara Solov'eva, Viktoria Davydova, Inna Krasikova, Irina Yermak. Mar Drugs. 2013 Jun 19; 11(6):2216-29. Marine compounds with therapeutic potential in gram-negative sepsis doi: 10.3390/md11062216.

8. Panda S K, Kumar S, Tupperwar N C, Vaidya T, George A, et al. Chitohexaose Activates Macrophages by Alternate Pathway through TLR4 and Blocks Endotoxemia. PLoS Pathog 8(5) 2012.

9. Berghe T V, Demon D, Bogaert P, Vandendriessche B, Goethals A, Depuydt B, et al. Simultaneous targeting of IL-1 and IL-18 is required for protection against inflammatory and septic shock. Am J Respir Crit Care Med. 189:282-91, 2014.

10. Eidt M V, Nunes F B, Pedrazza L, Caeran G, Pellegrin G, Melo D A, Possuelo L, Jost R T, Dias H B, Donadio M V, Oliveira J R. Biochemical and inflammatory aspects in patients with severe sepsis and septic shock: the predictive role of IL-18 in mortality. Clin. Chim. Acta. 453:100-106, 2016.

11. Stearns-Kurosawa D J, Osuchowski M F, Valentine C, Kurosawa S, Remick D G. The pathogenesis of sepsis. Annu Rev Pathol.; 6: 19-48, 2011.

12. Cai B, Deitch E A, Ulloa L. Novel insights for systemic inflammation in sepsis and hemorrhage. Mediators Inflamm. 642462, 2010.

13. Russell J A. Management of sepsis. N Engl J Med.; 355: 1699-1713, 2006.

14. Matsuda A, Jacob A, Wu R, Aziz M, Yang W L, Matsutani T, Suzuki H. Furukawa K, Uchida E, Wang P. Novel therapeutic targets for sepsis: Regulation of exaggerated inflammatory responses. J Nippon Med Sch.; 79: 4-18,2012.

15. Nolan A, Weiden M, Kelly A, Hoshino Y, Hoshino S, Mehta N, Gold J A. CD40 and CD80/86 act synergistically to regulate inflammation and mortality in polymicrobial sepsis. Am J Respir Crit Care Med. 177:301-308, 2008.

16. Flohé S B, Agrawal H, Schmitz D, Gertz M, Flohé S, Schade F U. Dendritic cells during polymicrobial sepsis rapidly mature but fail to initiate a protective Th1-type immune response. J Leukoc Biol.; 79: 473-481, 2006.

17. Roger P M, Hyvernat H, Ticchioni M, Kumar G, Dellamonica J. Bernardin G. The early phase of human sepsis is characterized by a combination of apoptosis and proliferation of T cells. J Crit Care.; 27: 384-393, 2012.

18. Paunel-Görgülü A, Kirichevska T, Lögters T, Windolf J, Flohé S. Molecular mechanisms underlying delayed apoptosis in neutrophils from multiple trauma patients with and without sepsis. Mol Med. 18: 325-335, 2012.

19. Wittebole, X., Castanares-Zapatero, D., & Laterre, P. F. (2010). Toll-like receptor 4 modulation as a strategy to treat sepsis. Mediators of Inflammation, 2010. https//doi.org/10.1155/2010/568396.

20. Opal. (2013). Effect of eritoran, an antagonist of MD2-TLR4, on mortality in patients with severe sepsis: The ACCESS randomized trial (JAMA—Journal of the American Medical Association (2013) 309, 11 (1154-1162)). JAMA—Journal of the American Medical Association, 310(3), 324. https://doi.org/10.1001/jama.2013.3460.

21. Kumar, P., Tyagi, R., Das, G., & Bhaskar, S. (2014). Mycobacterium indicus pranii and Mycobacterium bovis BCG lead to differential macrophage activation in Toll-like receptor-dependent manner. Immunology, 143(2), 258-268.https://doi.org/10.1111/imm.12306.

22. Sehgal, I. S., Agarwal, R., Aggarwal, A. N., & Jindal, S. K. (2015). A randomized trial of Mycobacterium w in severe sepsis. Journal of Critical Care, 30(1), 85-89. https://doi.org/10.1016/j.jcrc.2014.08.012.

23. Casadidio, C., Peregrina, D. V., Gigliobianco, M. R., Deng, S., Censi, R., & Di Martino, P. (2019). Chitin and chitosans: Characteristics, eco-friendly processes, and applications in cosmetic science, Marine Drugs, 17(6), https://doi.org/10.3390/md17060369.

24. Hyung, J. H., Ahn, C. .B., I L Kim, B., Kim, K., & Je, J. Y. (2016). Involvement of Nrf2-mediated heme oxygenase-1 expression in anti-inflammatory action of chitosan oligosaccharides through MAPK activation in murine macrophages. European Journal of Pharmacology, 793(May), 43-48. https://doi.org/10.1016/j.ejphar.2016.11.002.

25. Qiao, Y. Bai, X.-F., & Du, Y.-G. (2011). Chitosan oligosaccharides protect mice from LPS challenge by attenuation of inflammation and oxidative stress. International Immunopharmacology, 11(1), 121-127. https://doi.org/10.1016/j.intimp.2010.10.016.

26. Fuchs, K., Cardona Gloria, Y., Wolz, O., Herster, F., Sharma, L., Dillen, C. A., Täumer, C., Dickhöfer, S., Bittner, Z., Dang, T., Singh, A., Haischer, D., Schlöffel, M. A., Koymans, K. J., Sanmuganantham, T., Krach, M., Roger, T., Le Roy, D., Schilling, N. A., . . . Weber, A. N. (2018). The fungal ligand chitin directly binds TLR 2 and triggers inflammation dependent on oligomer size EMBO Reports, 19(12), 1-14. https://doi.org/10.15252/embr.201846065.

27. Rittirsch, D., Huber-Lang, M. S., Flierl, M. A., & Ward, P. A. (2009). Immunodesign of experimental sepsis by cecal ligation and puncture. Nature Protocols. https://doi.org/10.1038/nprot.2008.214.

28. Toscano, M. G., Ganea. D., & Gamero, A. M. (2011). Cecal ligation puncture procedure. Journal of Visualized Experiments: JoVE 51. https://doi.org/103.791/2860.

29. Oliveira-Nascimento, L., Massari, P., & Wetzler, L. M. (2012). The role of TLR2 ininfection and immunity. Frontiers in Immunology, 3(APR), 1-17. https://doi.org/10.3389/fimmu.2012.00079.

30. Yoshimura, T., & Takahashi., M. (2007). IFN-γ-Mediated Survival Enables Human Neutrophils to Produce MCP-1/CCL2 in Response to Activation by TLR Ligands. The journal of Immunology, 179(3), 1942-1949. https://doi.org/10.4049/jimmunol.179.3.1942.

31. Moon, S. K., Woo, J. I., Lee, H. Y., Park, R., Shimada, J., Pan, H., Gellibolian, R., & Lim, D. J. (2007). Toll-like receptor 2-dependent NF-κB activation is involved in nontypeable haemophilus influenzae-induced monocyte chemotactic protein 1 up-regulation in the spiral ligament fibrocytes of the inner ear. Infection and Immunity, 75(7), 3361-3372. https://doi.org/10.1128/IAI.01886-06.

32. Zhao, Q., Yin, L., Zhang, L., Jiang, D., Liu, L., & Ji, H. (2020). Chitoheptaose Promotes Heart Rehabilitation in a Rat Myocarditis Model by Improving Antioxidant, Anti-Inflammatory, and Antiapoptotic Properties. Oxidative Medicine and Cellular Longevity, 2020(LVIDd). https://doi.org/10.1155/2020/2394704.

33. Das, P., Panda, S. K., Agarwal, B., Behera, S., Ali. S. M., Pulse, M. E., Solomkin, J. S., Opal, S. M., Bhandari, V., & Acharya, S. (2019). Novel Chitohexaose Analog Protects Young and Aged mice from CLP Induced Polymicrobial Sepsis. Scientific Reports, 9(1), 1-12. https://doi.org/10.1038/s41598-019-38731-3.

34. Seemann, S., Zohles. F., & Lupp, A. (2017). Comprehensive comparison of three different animal models for systemic inflammation. Journal of Biomedical Science, 24(1), 1-17. https://doi.org/10.1186/s12929-017-0370-8.

35. Matsumoto, H., Ogura, H., Shimizu, K., Ikeda, M., Hirose, T., Matsuura, H., Kang, S., Takahashi, K., Tanaka, T., & Shimazu, T. (2018). The clinical importance of a cytokine network in the acute phase of sepsis. Scientific Reports, 8(1), 1-4. https://doi.org/10.1038/s41598-018-32275-8.

36. Hong, T. H., Chang, C. H., Ko, W. J., Lin, C. F., Liu, H. H., Chow, L. P., Huang, C. T., Yu, S. L., & Chen, Y. S. (2014). Biomarkers of early sepsis may be correlated with outcome. Journal of Translational Medicine, 12(1), 1-8. https://doi.org/10.1186/1479-5876-12-146.

37. Song, G. Y., Chung, C. S., Chaudry, I. H., & Ayala, A. (1999). What is the role of interleukin 10 in polymicrobial sepsis: Anti-inflammatory agent or immunosuppressant? Surgery, 126(2), 378-383. https://doi.org/10.1016/S0039-6060(99)70180-7.

38. Remick, D. G., Bolgos, G., Copeland, S., & Siddiqui, J. (2005). Role of interleukin-6 in mortality from and physiologic response to sepsis. Infection and Immunity, 73(5), 2751-2757. https://doi.org/10.1128/IAI.73.5.2751-2757.2005.

39. Re, F., & Strominger, J. L. (2004). IL-10 Released by Concomitant TLR2 Stimulation Blocks the Induction of a Subset of Th1 Cytokines That Are Specifically Induced by TLR4 or TLR3 in Human Dendritic Cells. The Journal of Immunology, 173(12), 7548-7555. https://doi.org/10.4049/jimmunol.173.12.7548.

40. Albrecht, V., Hofer, T. P. J., Foxwell, B., Frankenberger, M., & Ziegler-Heitbrock, L. (2008). Tolerance induced via TLR2 and TLR4 in human dendritic cells: Role of IRAK-1. BMC Immunology, 9. https://doi.org/10.1186/1471-2172-9-69.

41. Chen, A. S., Taguchi, T., Okamoto, H., Danjo, K., Sakai, K., Matahira, Y., Wang, M. W., & Miwa, I. (2005). Pharmacokinetics of chitobiose and chitotriose administered intravenously or orally to rats. Biological and Pharmaceutical Bulletin, 28(3), 545-548. https://doi.org/10.1248/bpb.28.545.

42. Moo-Yeal Lee, FigenVar, Yoshitsune Shin-ya, Toshio Kajiuchi, Ji-WonYang (1999). Optimum conditions for the precipitation of chitosan oligomers with DP 5-7 in concentrated hydrochloric acid at low temperature. Process Biochemistry, Volume 34, Issue 5, July 1999, Pages 493-500.

### OBJECTS OF THE INVENTION

An object of the present invention is to provide therapeutic and/or prophylactic molecules for combating sepsis.

An object of the present invention is to provide protein homolog of human HSP70 derived from nematode Setaria digitata, recombinant form or part thereof which modulates immune response in a mammal.

Yet another object of the present invention is to provide a subdomain of the HSP70 (C-Terminal Domain) as the active component of HSP70.

Another objective of the invention is to provide a therapeutic protein against sepsis that is effective even late after the onset of disease and improves the survival through immnomodulation in post septicaemia in mammals.

Still another object of the present invention is to provide a recombinant form of therapeutic protein homolog of human HSP70 derived from nematode Setaria digitata.

Yet another object of the present invention is to provide chito-oligosaccharide which is capable of treating and/or preventing sepsis, multiple organ dysfunction syndrome (MODS) or septic shock.

Still another object of the present invention is to provide purified oligosaccharide for sepsis intervention.

Still another object of the present invention is to provide a pharmaceutical composition comprising physiologically effective amount of proteins homolog of human HSP70 derived from nematode Setaria digitata and oligo saccharides for treatment and/or prevention of sepsis, multiple organ dysfunction syndrome (MODS) or septic shock.

Still another object of the present invention is to provide a method of treatment or prevention or prophylaxis of sepsis, MODS or septic shock in a mammal including human comprising administering an effective amount of protein obtained from the nematode Setaria digitata or chito-oligosaccharides or a combination thereof.

An object of the present invention is to provide oligosaccharide molecules which can be used alone or in combination with therapeutic proteins obtained from Setaria digitata in sepsis intervention.

### SUMMARY OF THE INVENTION

The present invention provides therapeutic and/or prophylactic molecules for combating sepsis. The invention provides proteins and oligosaccharides, capable of modulating inflammation in an animal model. The protein is a homolog of human HSP70 derived from nematode Setaria digitata. The invention provides proteins and oligosaccharides in immunomodulating inflammatory response in sepsis mammal. The invention demonstrates immunomodulatory role of proteins homolog of human HSP70 derived from nematode Setaria digitata and oligosaccharides in endotoxemia and CLP models of sepsis. The invention also provides a composition of therapeutic protein and oligosaccharide molecules for immunomodulation in sepsis. The invention further provides methods and pharmaceutical composition comprising proteins and/or oligosaccharides for modulating inflammation in subjects suffering from sepsis.

The present invention demonstrates effect of heat shock protein 70 (Hsp 70) from filarial worm for polymicrobial sepsis in CLP model.

The invention further studies therapeutic and/or prophylactic effects of acetylated chito-oligomers, specifically with the degree of polymerization of 6, 7 and 8 were studied in mice CLP model of sepsis.

The invention provides a novel function of the protein and establishes exogenous Hsp70 as a promising pharmacological agent for the therapeutic treatment of various types of sepsis.

The invention demonstrates an acetylated chito-oligomer Hepta N-acetyl chitoheptaose as an effective tool for sepsis.

The invention further provides a pharmaceutical composition for treatment, prophylaxis, of a subject suffering from sepsis or in need thereof.

The invention also provides a method of treatment, prophylaxis, of a subject suffering from sepsis or in need thereof.

The embodiments of the present invention are provided herein:

An embodiment of the present invention discloses a protein homolog of human HSP70 derived from nematode Setaria digitata which is capable of treating and/or preventing sepsis, multiple organ dysfunction syndrome (MODS) or septic shock.

In another embodiment the present invention discloses that the protein has 70-80% homology with the human HSP70.

In yet another embodiment the present invention discloses that the protein improves the survival through immunomodulation in post septicaemia in mammals.

In still another embodiment the present invention discloses that the protein immunomodulates by activation of TLR4 (Toll like receptor 4) and/or TLR2 receptors.

A further embodiment of the present invention discloses a chito-oligosaccharide which is capable of treating and/or preventing sepsis, multiple organ dysfunction syndrome (MODS) or septic shock wherein the chito-oligosaccharide is selected from a group comprising of a derivative of hexa-N-acetyl chitohexaose, hepta-N-acetyl chitoheptaose or octa-N-acetyl chitooctaose.

In another embodiment the present invention discloses the chito-oligosaccharide is preferably hepta-N-acetyl chitoheptaose.

In yet another embodiment the present invention discloses that the chito-oligosaccharide immunomodulates by activation of TLR4 (Toll like receptor 4) and/or TLR2 receptors.

In still another embodiment the present invention discloses that the chito oligosaccharide is administered before and/or after onset of symptoms of sepsis.

An embodiment of the present invention discloses a pharmaceutical composition comprising physiologically effective amounts of protein homolog of human HSP70 derived from nematode Setaria digitata and chito-oligosaccharide wherein the oligosaccharide is selected from the group comprising of a derivative of hexa-N-acetyl chitohexaose, hepta-N-acetyl chitoheptaose or octa-N-acetyl chitooctaose which is capable of treating and/or preventing sepsis, multiple organ dysfunction syndrome (MODS) or septic shock.

In another embodiment the present invention discloses that the pharmaceutical composition comprises the protein having 70-80% homology with the human HSP70.

In yet another embodiment the present invention discloses that the pharmaceutical composition comprises a protein which improves the survival through immunomodulation in post septicaemia in mammals.

In still another embodiment the present invention discloses that the pharmaceutical composition immuno-modulates by activation of TLR4 (Toll like receptor 4) and/or TLR2 receptors.

In a further another embodiment the present invention discloses that the pharmaceutical composition is effective for sepsis wherein said sepsis is mild sepsis, severe sepsis, infection symptoms or sepsis caused by burn, acute laryngopharyngitis, ulcerative colitis, IBS (Irritable Bowel syndrome), rheumatic arthritis, degenerative arthritis, acute hepatitis, or chronic hepatitis.

In yet another embodiment the present invention discloses that the pharmaceutical composition comprising the protein and/or the chito-oligosaccharide further comprising of, additives, binders and excipients or a combination thereof.

In still another embodiment the present invention discloses that the pharmaceutical composition further comprises an anti septic agent wherein said anti-septic agent is at least one selected from the group consisting of antibiotics such as amoxicillin, clavulanate, penicillin, quinolone, monobactam, aminoglycoside, cephalosporin, tetracycline, glycopeptides, carbapenem and the like; anti-inflammatory agents such as mefenamic acid, indomethacin, ibuprofen, piroxicam, diclofenac and the like; anti-fungal agent such as amphotericin, B. nystatin, griseofulvin, azole anti-fungal agent and the like; and anti-allergic agent such as cetirizine, fexofenadine, chlroropeniramine, and the like or a combination thereof.

An embodiment of present invention discloses a method of treatment or prevention of sepsis, MODS or septic shock in a mammal including human comprising administering an effective amount of protein homolog of human HSP70 derived from nematode Setaria digitata or chito-oligosaccharides wherein the chito-oligosaccharides is selected from the group comprising of a derivative of hexa-N-acetyl chitohexaose, hepta-N-acetyl chitoheptaose or octa-N-acetyl chitooctaose or a combination thereof together with a pharmaceutically acceptable carrier thereof to said mammal.

In another embodiment the present invention discloses that the method of treatment or prevention of sepsis, MODS or septic shock in a mammal including human comprises administering an effective amount of protein wherein the protein has 70-80% homology with the human HSP70.

In another yet embodiment the present invention discloses that the method of treatment or prevention of sepsis, MODS or septic shock in a mammal including human comprises administering an effective amount of chito-oligosaccharide wherein the chito-oligosaccharide is hepta-N-acetylchitoheptaose.

A further embodiment of present invention discloses a synthetic or recombinant nucleic acid encoding a protein homolog of human HSP70 derived from nematode Setaria digitata or a chito-oligosaccharide selected from the group comprising of a derivative of hexa-N-acetyl chitohexaose, hepta-N-acetyl chitoheptaose or octa-N-acetyl chitooctaose.

In still another embodiment the present invention discloses that the nucleic acid encodes a protein having 70-80% homology with the human HSP70.

An embodiment of present invention discloses a use of a protein homolog of human HSP70 derived from nematode Setaria digitata or a recombinant form or a derivative thereof and/or chito-oligosaccharide wherein the chito-oligosaccharide is selected from a group comprising of a derivative of hexa-N-acetyl chitohexaose, hepta-N-acetyl chitoheptaose or octa-N-acetyl chitooctaose for preparation of therapeutic agent for treatment, prophylaxis and/or prevention of sepsis, MODS or septic shock in a mammal including human.

In another embodiment the present invention discloses a use of a protein homolog of human HSP70 derived from nematode Setaria digitata or a recombinant form or a derivative thereof wherein the protein has 70-80% homology with the human HSP70.

An yet another embodiment of the present invention discloses a health functional food comprising protein homolog of human HSP70 derived from nematode Setaria digitata or a recombinant form or a derivative thereof, and/or chito-oligomer wherein the chito-oligosaccharide is selected from a group comprising of a derivative of hexa-N-acetyl chitohexaose, hepta-N-acetyl chitoheptaose or octa-N-acetyl chitooctaose, or a combination thereof as an active ingredient for alleviating or preventing sepsis, MODS or septic shock.

### BRIEF DESCRIPTION OF THE DRAWINGS

FIG. 1A: The size exclusion profile for AgW

FIG. 1B: Effect of native proteins on THP-1 cells.

FIG. 1C: Effect of P1 and rest of the proteins pooled together (Pp) on induction of cytokines. The figure shows that P1 activated the THP-1 monocytic cells while the rest of the proteins pooled together (Pp) had minimal induction of cytokines.

FIG. 2: Effect of different doses of native protein on mice survival in CLP model.

FIG. 3: Effect of native protein, P1 (G2, LPS+D1A1) on mice survival in endotoxemia model.

FIG. 4: Effect of chito-oligosaccharides in CLP model.

FIGS. 4A-4B: Effect of LPS, GA132 and GA120 on induction of cytokines.

FIGS. 4C-4F: Effect of GA132 on cytokine induction on primary human blood derived immune cells (PBMCs).

FIGS. 4G-4H: Comparative effect of antibiotic alone and GA132 on Balb/c and C57B1/6 mice models, respectively.

FIGS. 4I-4J: Characterization of GA132 by HPLC and MALDI-TOF, respectively.

FIGS. 4K-4M: Effect of chito-oligosaccharides on various surface receptors of HEK-blue cell lines.

FIGS. 5A and 5B: Percent survival of mice in CLP model treated with various oligosaccharides.

FIG. 5C: Comparative effect of varying concentration of different oligosaccharides and antibiotic on induction of cytokines and MCP.

FIG. 5D: Comparative effect of varying concentration of different oligosaccharides and antibiotic on induction of cytokines and MCP with time.

FIG. 6: Effect of P1, Pp, LPS, P1+LPS, Pp+LPS on induction of cytokines and MCP.

FIG. 7: Effect of P1 on various surface receptors. P1 fraction tested on reporter cell lines where TLR2, TLR4 or TLR3 is expressed in HEK293 cells and activation of the receptors is observed by NP-kB and AP-1 promoter driven SEAP enzyme secretion. P1 activates the surface expressed receptors TLR2 and TLR4 while no activation was seen in TLR3 cells and control cells expressing only the reporter construct.

FIG. 8: Effect of P1 and LPS on induction of various cytokines owing to level of expression of mRNA produced.

FIG. 9: Effect of combination of protein and chito-oligosaccharides on immunomodulation.

### DETAILED DESCRIPTION OF THE INVENTION

Setaria digitata is a filarial worm that infects cattle and is used as a model worm to study nematode infection. Panda et al; 2012 showed that the fraction made from the total soluble worm lysate has affinity to WGA (Wheat germ agglutinin) lectin (termed as AgW). This fraction contains multiple glycoproteins and was able to bind to TLR4. The AgW fraction provides protection to mice in endotoxemia model that is limited to simulating only gram-negative infections. However, the fraction was not purified further and the underlying active component was not identified.

In the present invention, the native protein molecule was obtained from Setaria digitata and its effect was studied in preclinical models of sepsis namely, endotoxemia model and in CLP (a gold standard model of sepsis where the poly-microbial sepsis is simulated). The protein showed promising immunomodulatory results in both the models of sepsis. The recombinant version of the native protein was expressed in E. coli and process for large-scale production was optimised. The effect of these proteins was also studied on survival rate in septic mice in both the endotoxemia and CLP models.

The invention further demonstrates therapeutic effects of acetylated chito-oligomers, specifically with the degree of polymerization of 6, 7 and 8 in mice CLP model of sepsis. The invention further provides pharmaceutical composition for treatment, prophylaxis, therapy, medicinal of a subject suffering from sepsis or in need thereof. The invention also provides a method of treatment, prophylaxis, therapy, medicinal of a subject suffering from sepsis or in need thereof.

The present invention is described in greater detail with examples below, which are for illustrative purposes and should in no way be construed as limiting the invention.

### EXAMPLE 1

### Isolation of Native Protein P1

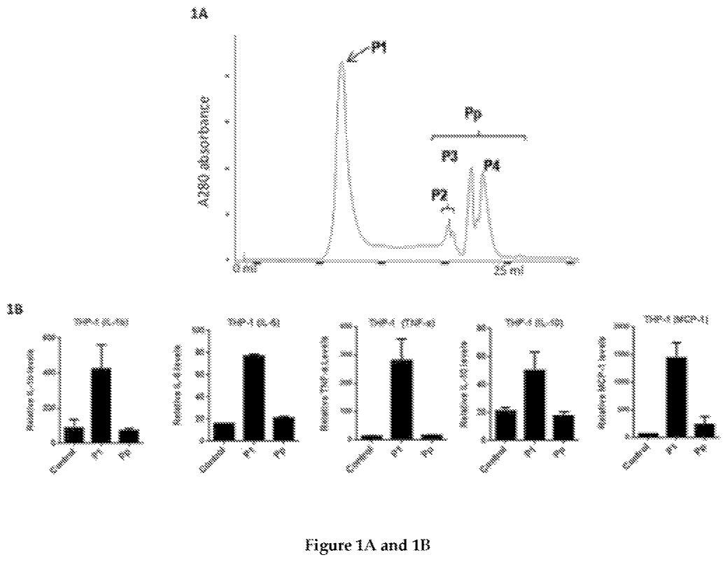

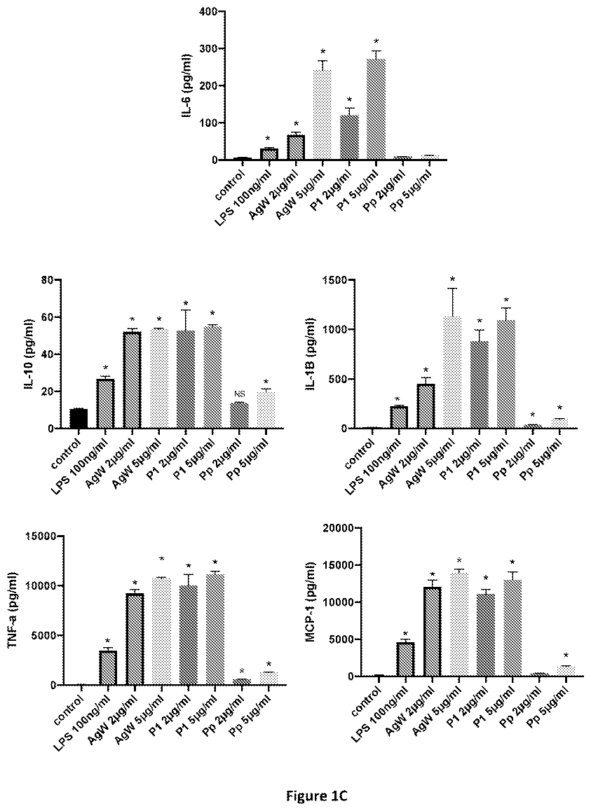

The Setaria digitata (Nematode) used for the purpose of this invention was peritoneal dwelling adult female filarial parasites obtained from cattle in a local abattoir, attached to the local zoological park at Nandankanan, Bhubaneswar after obtaining necessary approval from zoo authorities. The AgW was fractionated on G75 size exclusion column where a major peak, P1 was seen apart from low peaks, P2, P3 and P4. As the quantity of other peaks was low they were pooled together as Pooled Peaks or Pp fraction (FIG. 1A). The individual fractions were investigated for their ability to bind with and activate TLR4 and TLR2 immune receptors. There was significant seasonal variation in the AgW composition and the most potent fraction was named as P1 (peak 1).

The P1 fraction was found to be a pure glycoprotein and identified as Setaria digitata Heat Shock Protein 70 (SD-HSP70) by Mass Spectrometer base proteomic analysis. The recombinant version of SD-HSP70 was expressed in E. coli for scalability reasons. The E. coli cells used for the purpose of this invention were E. coli DH5alpha cells commercially procured from Thermo

### Heat Shock Protein 70

The Heat shock protein 70 [Setaria digitata] has accession number as GenBank: AAD13154.1 and has a sequence as shown below (SEQ. ID NO. 1)

MSKNAIGIDLGTTYSCVGVFMHGKVEIIANDQGNRTTPSYVAFTDTERLIG
DAAKNQVAMNPHNTVFDAKRLIGRKFDDGSVQSDMKHWPFKVMNAGGGKPK
VQVEYKGETKTFTPGEISSMVLVKMKETAEAFLGHAVKDAVITVPAYFNDS
QRQATKDSGAIAGLNVLRIINEPTAAAIAYGLDKKGHGERNVLIFDLGGGT
FDVSILTIEDGIFEVKSTAGDTHLGGEDFDNRMVNHFVAEFKRKHKKDLAS
NPRALRRLRTACERAKRTLSSSSQASIEIDSLFEGIDFYTNITRARFEELC
ADLFRSTMDPVEKALRDAKMDKAQVHDIVLVGGSTRIPKVQKLLSDFFSGK
ELNKSINPDEAVAYGAAVQAAILSGDKSEAVQDLLFVDVAPLSLGIETAGG
VMTALIKRNTTIPTKTSQTFTTYSDNQPGVLIQVYEGERAMTKDNNLLGKF
ELSGIPPAPRGVPQIEVTFDIDANGILNVSAQDKSTGKQNKITITNDKGRL
SKDEIERMVQEAEKYKADDEAQKDRIAAKNALESYAFNMKQTIEDEKLRDK
LSEEDKKKIQEKCDETVRWLDGNQTAEKDEFEHRQKELEAVSNPIITKLYQ
SAGGMPGGMPGGMPGGAPGGGSGGSGPTIEEVD

### HSPA1A Protein [Homo Sapiens HSP70]

The Heat shock protein HSPA1A [Homo sapiens] has accession number as >AAH18740.1 HSPA1A protein [Homo sapiens] and has a sequence as shown below (SEQ. ID NO. 2)

MAKAAAIGIDLGTTYSCVGVFQHGKVEIIANDQGNRTTPSYVAFTDTERLI
GDAAKNQVALNPQNTVFDAKRLIGRKFGDPVVQSDMKHWPFQVINDGDKPK
VQVSYKGETKAFYPEEISSMVLTKMKEIAEAYLGYPVTNAVITVPAYFNDS
QRQATKDAGVIAGLNVLRIINEPTAAAIAYGLDRTGKGERNVLIFDLGGGT
FDVSILTIDDGIFEVKATAGDTHLGGEDFDNRLVNHFVEEFKRKHKKDISQ
NKRAVRRLRTACERAKRLSSSTQASLEIDSLFEGIDFYTSITRATRFEELC
SDLFRSTLEPVEKALRDAKLDKAQIHDLVLVGGSTRIPKVQKLLQDFFNGR
DLNKSINPDEAVAYGAAVQAAILMGDKSENVQDLLLLDVAPLSLGLETAGG
VMTALIKRNSTIPTKQTQIFTTYSDNQPGVLIQVYEGERAMTKDNNLLGRF
ELSGIPPAPRGVPQIEVTFDIDANGILNVTATDKSTGKANKITITNDKGRL
SKEEIERMVQEAEKYKAEDEVQRERVSAKNALESYAFNMKSAVEDEGLKGK
ISEADKKKVLDKCQEVISWLDANTLAEKDEFEHKRKELEQVCNPIISGLYQ
GAGGPGPGGFGAQGPKGGSGSGPTIEEVD

### EXAMPLE 2

### Effect of Native Protein on Inflammation

THP-1 cells (human acute monocytic leukemia cell line) were procured from ATCC (Cat. No# ATCC® TIB-202™). The THP-1 cells were treated with the P1 and Pp to check for any pro-inflammatory activity. FIG. 1B illustrates that the protein is highly active in promoting the secretion of pro-inflammatory cytokines. The protein was found to be consistently activating the TLR4 and TLR2 receptors on reporter cells as well as monocytes and primary immune cells. Protein treatment in THP-1, human monocytic cells promote secretion of cytokines including IL-1b, IL-6, IL-10, TNF-α and MCP-1 (FIG. 1C) and reduces the alternate activation marker, CLEC10A or CD301. The protein was found to activate pro-inflammatory signaling through the TLRs in an NF-kb and AP-1-dependent manner (FIG. 7).

### EXAMPLE 3

### Effect of Native Protein on Survival Rate in CLP Model

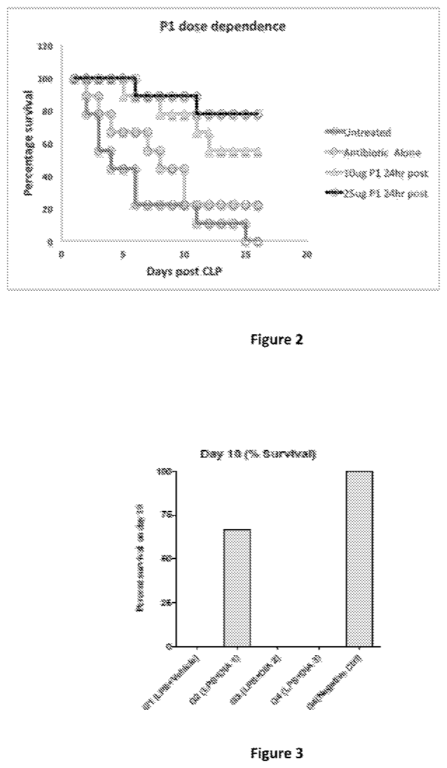

The native protein showed promising results in increasing survival in septic mice in the CLP model. About 80% of mice that received 15-35 μg of protein and about 60% mice that received 1-20 μg of protein survived even up to 16 days. More preferably, 80% of mice that received 25 μg of protein and 60% mice that received 10 μg of protein survived even up to 16 days. All the mice in the control group had died in the same duration. Hence, it appears that the protein can protect mice from polymicrobial sepsis as evidenced in the CLP animal model even 24 hours post-onset and a single dose of 25 μg was sufficient for protecting the septic mice (FIG. 2). Similar results were seen in the endotoxemia model.

### EXAMPLE 4

### Effect of Native Protein on Survival Rate in Endotoxemia Model

The native protein showed promising results in increasing the survival in septic mice in endotoxemia model (FIG. 3). In this model, post LPS Injection (i.e., 6 hours) animals were treated with vehicle and test agents amongst different groups G1-G5 as specified in figure. G2 (LPS+DIA-1) was treated with the P1 protein while G3 and G4 were other test protein used as experimental controls. All the animals survived up to ten days and the group treated with P1 showed 66% survival. All surviving animals were found to be apparently normal in G2 (LPS+DIA1) and Vehicle control (i.e., G5) Group.

### EXAMPLE 5

### Effect of Native Protein on of IL-18 Levels In Vitro

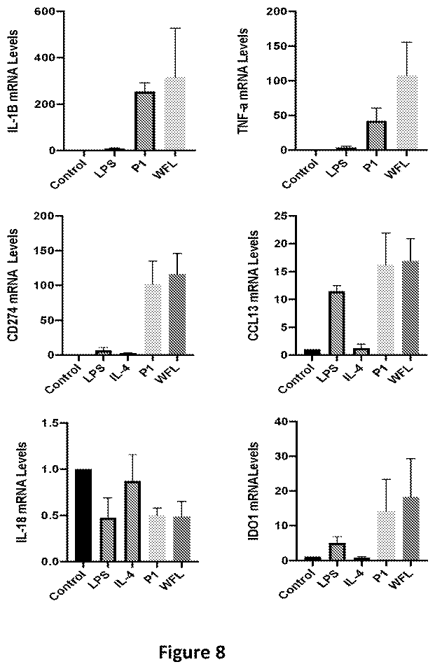

Increased levels of IL-18 have also been associated with higher mortality in sepsis. In endotoxemia mice models IL-18 knockout mice show better survival than the wild type indicating a role of IL18 in disease severity (Berghe T V, 2014 Eidt M V, 2016). The protein also altered the levels of sepsis-related marker like in vitro. The protein treatment of monocytes led to more than 60% reduction in IL-18 transcript levels (FIG. 8).

### EXAMPLE6

### Cell Lines and Primary Cells

HEK-Blue TLR2, HEK-Blue TLR3 and HEK-Blue TLR4 reporter cells expressing SEAP (secreted embryonic alkaline phosphatase) enzyme under NF-kb and AP-1 promoter (Cat. no.# hkb-htlr2, hkb-htlr3 and hkb-htlr4) were purchased from InvivoGen (USA). Human Peripheral blood mononuclear cells (PBMCs) (Cat. no.# CL003-25) were purchased from Himedia laboratories (India). HEK based cells and PBMCs were cultured DMEM and RPMI media, respectively. The cells were maintained at 37° C. incubator with 5% CO₂. The culture media was supplemented with 10% Fetal Calf Serum (Gibco) along with standard antibiotics. HEK-Blue selection antibiotics (InvivoGen, Cat. no.# hb-sel, ant-zn and ant-bl) were added as per manufacturers' instructions.

### Chemicals and Reagents

Hexa-N-acetyl Chitohexaose (Cat. no.# 56/11-0050), Hepta-N-acetyl Chitoheptaose (Cat. no.# 57/11-0010) and Octa-N-acetyl Chitooctaose (Cat. no.# 57/12-0010) termed 6-mer, 7-mer and 8-mer respectively were purchased from Isosep, Sweden. All other chemicals were purchased from Sigma. Chito-oligomer mixtures were generated by previously used method using acid hydrolysis with modifications (Moo-Yeal et al., 1999). Briefly, chitosan (Sima-Aldrich cat no. 448877) was hydrolyzed with cone. HCl by heating at 70-75° C0.for 30-60 minutes. The hydrolyzing step was carried preferably at 72° C. for 45 minutes. Same amount of water was added to the reaction mixture and kept at about −20° C. for about two days. The precipitate was washed with chilled ethanol and acetone followed by drying under vacuum. The dried precipitate was acetylated using Ac₂O in presence of triethyl amine. To deacetylate hydroxyl groups, the reaction mixture was treated with methanolic NaOH. The mass profiling of the sample suggested the presence of 3-10 mers of N-acetyl glucosamine. Different fractions were generated on Biogel-P4 (Bio-Rad and Cat. No. 1504124) and tested on reporter cells.

### TLR Reporter Assays

2×10⁴ HEK-Blue reporter cells were seeded, in each well of a 96 well plate, in HEK-Blue detection media (InvivoGen, Cat. no.# hb-det3) along with the 50 μg/ml of the chito-oligomers. 10 ng/ml Pam3CSK4 (InvivoGen, Cat. no.# tlrl-pms.), 100 ng/ml Poly I:C (InvivoGen, Cat. no.# tlrl-pic.), and 10 ng/ml LPS-EK (InVivoGen, Cat. no.# tlrl-eklps), were used as a positive control for HEK-Blue TLR2, TLR3 and TLR4 cells respectively. The treated cells were incubated at about 37° C. for about 16 hours and absorbance (O.D.) was measured at 620 nm in either TECAN Infinite® 200 PRO or Thermo Scientific Varioskan LUX multimode reader.

### Mice and CLP Study

The CLP study with 7-9 week old C57BL/6 or BALB/c males was conducted, at animal house facility at TheraIndx Lifesciences Pvt. Ltd. (Bangalore, India) and Institute of Life Sciences (Bhubaneswar, India) respectively, in accordance with ethical practices laid down in the CPCSEA guidelines for animal care and use (CPCSEA, 2003). The Institutional Animals Ethics Committee (IAEC) of the test facility approved the studies. The CLP was performed as described before (Rittirsch et al., 2009; Toscano et al., 2011). Sham control mice had undergone surgery with ceacal manipulations without ligation and puncture. About 500 μl saline was administered subcutaneously immediately after surgery. Tramadol (about 20 mg/kg) was injected subcutaneously for post-operative analgesia. BALB/c mice were injected with single IP close of about 500 μg GA132 (GA is the internal code which has been used by the inventors of the present invention) while C57BL/6 mice received single IP dose of about 300 μg or about 700 μg GA132 6 hours post CLP. A separate group of C57BL/6 mice received single IP dose of about 250 μg of 6-mer, 7-mer or 8-mer at either 6 hour or 24 hour post CLP. All groups except the CLP alone (control) received a single dose of standard antibiotics (amoxicillin and clavulanate) 6 hour post CLP. Control group was injected with saline solution. Total of 92 C57BL/6 and 18 BALB/c mice were used in the study.

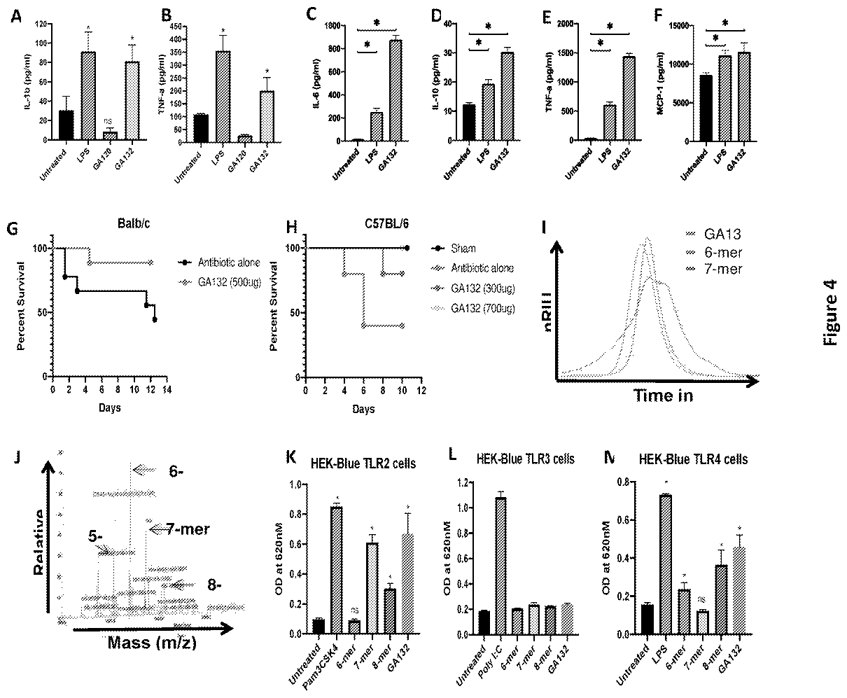

It was observed that animals were protected in all the treated groups, significant mortality reduction was observed with the administration of single dose of 200-250 μg chitoheptaose 6 hours and 24 hours post-CLP till day 7 of the experiment (FIG. 4).

### Bio-Plea Multiplex Assays

The Mutiplex immunoassay for Human and Mouse IL-1b, IL-6, IL-10, TNF-α and MCP-1 were purchased from Bio-Rad. For human cytokines, 1×10⁶ PBMCs were seeded in 24 well plate a day before and treated with 100 ng/ml LPS or 50 μg/ml GA132 for 24 hours. The culture supernatant from three independent biological replicates was collected and used for the assay without dilution. For mice cytokines, the plasma samples from mice were collected 6 or 48 hours post treatment and diluted in PBS (Phosphate Buffered Saline) in 1:2 before assay. The assay was performed as per manufacturers' method and the beads were read and analyzed in Bio-Plex® MAGRIX™ Multiplex Reader (Bio-Rad) (https://www.bio-rad.com/webroot/web/pdg/lsr/literature/10014905.pdf).

### HPLC Analysis

The samples were prepared in Ammonium acetate buffer (0.2 M). About 10 μg of GA132 or purified standards of Hexa-N-acetyl Chitohexaose (Cat. no.# 56/11-0050), Hepta-N-acetyl Chitoheptaose (Cat. no.# 57/11-0010) were analyzed with TSK-Gel (Cat. no.# G2000SWx.) on HPLC (Agilent Technologies 1260 Infinity). The change in refractive index was observed over time for different samples. The data was plotted using OpenLAB Control (FIG. 4I).

### Maldi-TOF/TOF Mass Spectrometry Analysis

For sample analysis, about 1.5 μl of 10-mg/mL α-CHCA (α-cyano-4-hydroxycinnamic acid) matrix solution was mixed with about 1.5 μl of each sample. From the resulting solution, about 1 μl was spotted onto the 384Opti-TOF 123 mm×81 mm SS target plate (AB Sciex). After drying at room temperature, spotted samples were analysed using a ABSCIEX TOF/TOF 5800 mass spectrometer (Applied Bio systems, USA) equipped with a 200 Hz, 355 nm Nd: YAG and acquired MS spectra followed by External mass calibrations for reflector mode were performed using a calibration mix (Cal mix TOF/TOF, Sciex) containing des-Arg1-bradykinin (m/z 904.468), Angiotensin I (m/z 1296.685), Glu1-fibrinopeptide B (m/z 1570.677), ACTH clip 1-1.7 (m/z 2093.086), ACTH clip 18-39 (m/z 2465.199), and ACTH clip 7-38 (m/z 3657.929) diluted to the manufacturers specifications (1-3 pmoL/μL each). The identified masses are exported as peek list (FIG. 4J).

### Statistical Analysis

Experimental data were analyzed using GraphPad Prism 8 (GraphPad Software, Inc.). Kruskal-Wallis test and one way ANOVA test was used for analysis of cytokines and reporter assay values. Log-rank (Mantel-Cox) test was utilized for survival analysis. P-values, P<0.05 were considered statistically significant and are denoted by throughout if calculated P-values were lower than P=0.05.

### Chita-Oligomers has Immunomodulatory Properties

Different combinations of chito-oligomers were produced from chitosan by varying time and concentration of acid hydrolysis followed by subsequent fractionation/purification. These fractions were screened using HEK-Blue TLR reporter assays NF-kb and AP-1 driven SEAP activity was measured (unpublished). Based on the reporter cell assays, one of the selected active fractions from the screening, GA132 and its deacetylated version, GA120 were tested on monocytic cell line. THP-1 cells were stimulated with equal amounts of either GA132 or GA120 and the levels of IL-1b (FIG. 4A) and TNF-α (FIG. 4B) were measured in the culture after 24 hours. The results indicated that GA132 was able to induce the secretion of both IL-1b and TNF-α, while the deacetylation abrogated this activity (FIGS. 4A and 4B). These results were consistent with previous finding, where treatment of long chain acetylated chito-oligomers, C10-15 and zymosan with hot alkali based deacetylation led to reduced NF-kb activation in TLR2-transfected HEK293T cells (Fuchs et al., 2018).

The TLR2/4 dependent activation of NF-kb and AP-1 leads to activation of immune cells and secretion of cytokines like IL-1b, IL-6, TNF-α and MCP-1, IL-10 in response to different stimuli (Oliveira-Nascimento et al., 2012; Yoshimura & Takahashi, 2007) (Moon et al., 2007). IL-10 secretion is associated with alternate activation of macrophages and has shown to be protective in sepsis endotoxemia models (Panda et al., 2012). To further validate the observations from reporter cells and monocytic cell line, the effects of GA132 on cytokine induction were further studied on primary human blood derived immune cells (PBMCs). PBMCs were treated for 24 hours, where GA132 treatment showed potent induction of IL-6, IL-10, TNF-α and MCP-1 (FIGS. 4C, 4D, 4E and 4F). These results confirmed that GA132 was indeed capable of immunomodulation of innate immune response with simultaneous secretion of both pro and anti-inflammatory cytokines and may help in restoring the immune homeostasis.

### GA132 protects mice in CLP model

The immunomodulatory properties of GA132 were later assessed in the CLP model, a gold standard model for studying sepsis in mice (Toscano et al., 2011). The CLP model was developed in Balb/c and C57BL/6 mice as both have different susceptibilities to infection and the effect of GA132 was tested post-surgery. In Balb/c mice, the group treated with about 500 μg (−15 mg/kg) GA132 along with standard antibiotic treatment showed improved survival as compared to the group that received only the antibiotics (FIG. 4G). Based on this results, one lower dosage of about 300 μg (−10 mg/kg) and higher dosage of about 700 μg (˜20 mg/mg) GA132 was evaluated in C57BL/6 CLP mice, where both the dosage were found to be effective and higher dosage of GA132 showed survival in all tested mice (FIG. 4H). In the in-vivo studies GA132 mixture showed significant protection in both the mice models and the role of individual components of GA132 was evaluated in later experiments.

### Characterization of GA132

To determine the composition of GA132, it was subjected to HPLC (FIG. 4I) MALDI-ToF (FIG. 4J) analysis. The HPLC results showed that the GA132 had an overlapping peaks corresponding to Hexa-N-acetyl Chitohexaose (6-mer) and Hepta-N-acetyl Chitoheptaose. (7-mer) along with small chain oligomers (FIG. 4I). The MALDI-ToF analysis showed presence of completely acetylated chito-oligomers with chain length of 4-8. The major species were identified with mass of 1056.3, 1259.5, 1462.4 and small amounts of 1665.7 corresponding to sodium salt of Penta N-acetyl Chitopentaose (5-mer), Hexa N-acetyl Chitohexaose (6-mer), Hepta N-acetyl Chitoheptaose (7-mer) and Octa N-acetyl Chitooctaose (8-mer respectively (FIG. 4J).

Hepta N-acetyl Chitoheptaose (7-mer) and Octa N-acetyl Chitooctaose (8-mer) have different effects on TLR2 and TLR4 based NE-kb and AP-1 activation.

Previous studies have indicated that a chain length of 6 or more is required for binding to TLR2 (Fuchs et al., 2018) and the chain length of the Chito-oligomer plays an important role in bio-activity (Panda et al., 2012; Zhao et al., 2020).

As GA132 showed presence of 6, 7 and small amounts of 8 mer, the activity on individual purified components was assessed. For this, HPLC purified 6-mer, 7-mor and 8-mer were tested for their effect on different TLRs using specific HEK-Blue reporter cells (FIGS. 4K, 4L and 4M). The results indicated that GA132, 7-mer and 8-mer activated TLR2, while 6-mer had no significant effect on TLR2 based NF-kB and AP-1 activation (FIG. 4K). In another study, the chitohexaose derivative, AVR-25 did not show any binding affinity to TLR2 (Das et al., 2019). Surprisingly, the activity of 7-mer was significantly higher than 8-mer in TLR2 reporter cells. The effect of GA132 and purified Chito-oligomers was also tested on HEK-Blue TLR4 reporter cells, where surprisingly GA132, 6-mer and 8-mer had some degree of activation while 7-mer had not effect (FIG. 4M). This study explored and delineated TLR specificity for different chain length of acetylated chito-oligomers. These oligomers were also tested on HEK-Blue TLR3 reporter cells as a control where no activation was observed (FIG. 4K).

### Hepta N-acetyl Chitoheptaose (7-Mer) Improves Survival in CLP Model

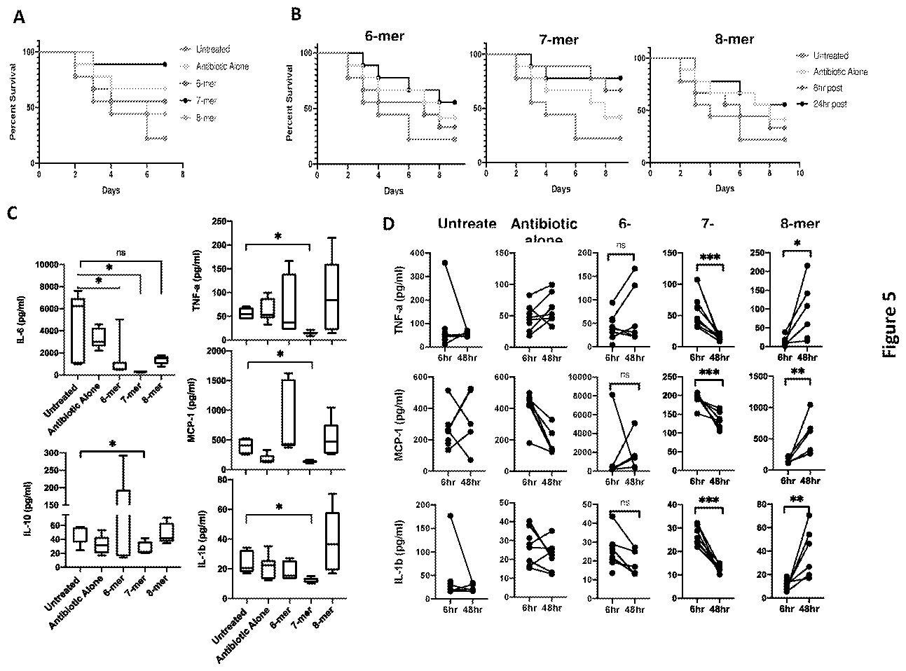

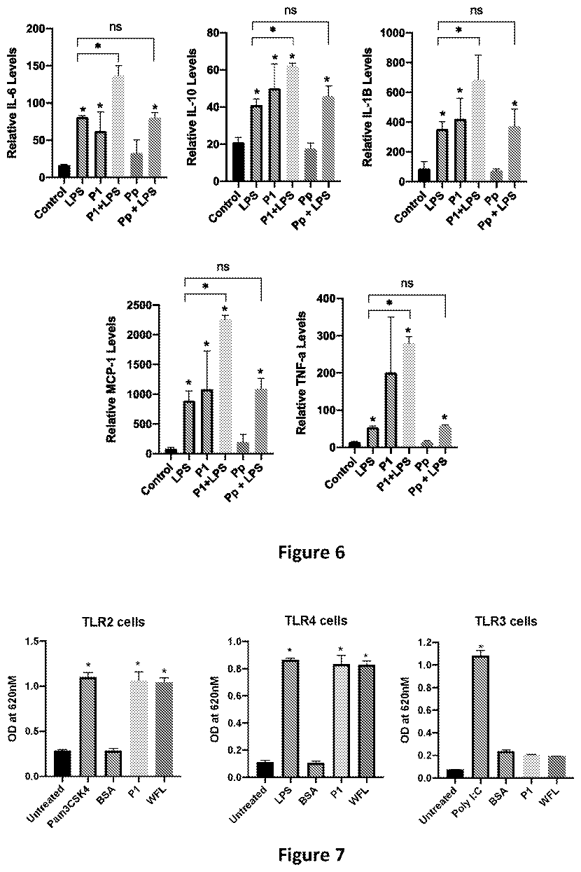

The protective effects of purified 6-mer, 7-mer and 8-mer were then assessed in the CLP model. Purified oligomers were used along with standard of care antibiotics and the intervention was either done at 6 hours or 24 hours post-surgery (FIGS. 5A to 5C). At 6 hours intervention, the survival in different group was evaluated over 7 days, where 7-mer showed around 90% protection as compared to 60% survival in ‘Antibiotic—alone’ group and 20% survival in untreated group. Surprisingly, 6-mer and 8-mer showed no additional protection over ‘Antibiotic-alone’ group. 8-mer treated group had only 40% survival up to 7 days, indicating that intervention with 8-mer along with the standard of care is rather detrimental for survival. This was different from previous observation where chitohexaose, CHTX was found to protect mice in endotoxemia model (Panda et al., 2012). This difference could be due to use of a different model as endotoxemia model utilized only a purified LPS molecule whereas CLP model simulates polymicrobial sepsis where multiple pathways may get involved and studies have indicated that Mice exposed to either LPS or peritoneal contamination and infection (PCI) recovered after 72 hours whereas, CLP induced comparatively more protracted course of inflammation (Seemann et al., 2017). In another study, a derivative of chitohexaose was found to protect young and aged mice in the CLP model (Das et al., 2019), wherein the effects of un-derivatized chitohexaose were not explored. To assess the effect of intervention at later stages, a single dose of the purified chito-oligomer was given at 24 hours and the mortality was scored and compared to the group when treated at earlier time point of 6 hours (FIG. 5B). Intervention with 7-mer showed 80% survival up to 7 days, while 6-mer and 8-mer showed no change as compared to the ‘Antibiotic alone’ group.

### Hepta N-acetyl Chitoheptaose (7-Mer) Reduces Cytokine Storm

Recent clinical studies have indicated that a cytokine network of IL-6, IL-8, MCP-1 and IL-10 plays a pivotal role in acute phase of sepsis and higher levels of IL-6, TNF-α, IL1-b and MCP-1 were associated with higher mortality in sepsis patients (Matsumoto et al., 2018)(Hong et al., 2014). Treatments where levels of these cytokines were reduced, significantly improved survival in mice models (Das et al., 2019; Panda et al., 2012; Song et al. 1999). Hence, plasma levels of these cytokines at 6 hours and 48 hours post treatment were analyzed from CLP mice treated with 6-mer 7-mer and 8-mer where, the treatment was given along with the standard of care antibiotics at 6 hours post CLP.

As earlier studies indicated elevation in IL-6 levels at early stages in the CLP model (Remick et al., 2005), the IL-6 levels were measured at 6 hours post intervention. The results showed a significant decrease in 6-mer and 7-mer treated mice (FIG. 5C). The IL-6 levels were also reduced in 8-mer treated mice when compare to untreated mice but the difference was not found to be significant in statistical analysis (FIG. 5C). Analysis of IL-10, TNF-α, IL-1b and MCP-1 at 48 hours post-surgery showed significant reduction only in 7-mer treated group when compared to the untreated group (FIG. 5D). In the 6-mer and 8-mer treated group, variability in levels of cytokines from individual mice was much higher while, in 7-mer treated group the standard deviation between individual mice was minimal (FIG. 5D).

The inventors of this invention further analyzed the effect of 6-mer, 7-mer and 8-mer on progression of TNF-α, MCP-1 and IL-1b over time in individual mice from different groups. The plasma was collected at 6 hours and 48 hours post treatment and levels of these cytokines were measured and compared within the individual mice over time (FIG. 5D). Surprisingly, the results showed significant reduction in the levels of TNF-α, IL-1b and MCP-1 over time in only 7-mer treated group, while 8-mer treatment led to increase in the levels of these cytokines in same time period. 6-mer treatment had no effect on the levels on these cytokines (FIG. 5D). This sustained increase in pro-inflammatory cytokines may explain the differences obtained in the survival study where 7-mer showed improved recovery while 8-mer treatment reduces survival (FIG. 5A).

Previous studies using chito-oligosaccharides were performed using chitosan, the deacetylated form of chitin while current study was focused on the completely acetylated version of these chito-oligomers (Qiao et al., 2011). In this study, results clearly indicate that incremental change in chain length of chito-oligomer is not directly related to incremental increase in the bioactivity as previously presumed (Panda et al., 2012). Similar observation was made recently in another disease model where the effect of different chain chito-oligomers was studied in Myocarditis Model where 7-mer showed better activity in-vivo than 6-mer and 8-mer (Zhao et al., 2020). Here, the response of the 7-mer on the pro-inflammatory cytokine release under diseased condition in-vivo was found to be different than the in-vitro conditions, where the experiments were done on healthy immune cells. Previous studies have indicated that in human dendritic cells, rapid IL-10 release post TLR 2 stimulation blocks the induction of a subset of Th1 cytokines those are specifically induced by TLR4 or TLR3 and cross-talk between different TLRs may modify the primary responses of TLR to their agonist (Re & Strominger, 2004). Also, sub-activating doses of TLR2 ligands were found to induce tolerance in the dendritic cells in an IRAK1 dependent manner (Albrecht et al., 2008). These may explain surprising observations around the switch in the activity from promoting the cytokine production to preventing over stimulation; however it needs further analysis to identify the exact mechanism. Previously observed differences in absorption and bio-availability of chito-oligomers based on the size (Chen et al., 2005; Zhao et al., 2020) along with capability to stimulate different TLRs may explain possible difference between 7-mer and 8-mer in the in-vivo conditions (FIGS. 4K, 4L and 4M).

Provided easy availability of raw material for its synthesis, GA132 fraction and its main component, Hepta N-acetyl Chitohoptaose can be easily produced at large scale for therapeutic applications in sepsis and other diseases, where immunomodulation is required.

### EXAMPLE 7

### Effect of Combination of Protein and Chito-Oligosaccharides on Immunomodulation

The effect of combination of protein obtained from Filarial worm (Setaria digitata) and chito-oligosaccharides on immunomodulation was further analyzed. In order to study effect of the combination, the inventors treated THP-1 cells with protein and chito-oligomer alone and then with their combination. The THP-1 cells were treated in-vitro with 100-250 ng/ml of HSP70 protein; 50-150 μg/ml 7-mer (Hepta N-acetyl Chitoheptaose); and a combination of 100-250 ng/ml of full length HSP70 protein and 50-150 μg/ml of 7-mer (Hepta N-acetyl Chitoheptaose). The secreted cytokine levels were measured by Bioplex assay after 24 hours of treatment.

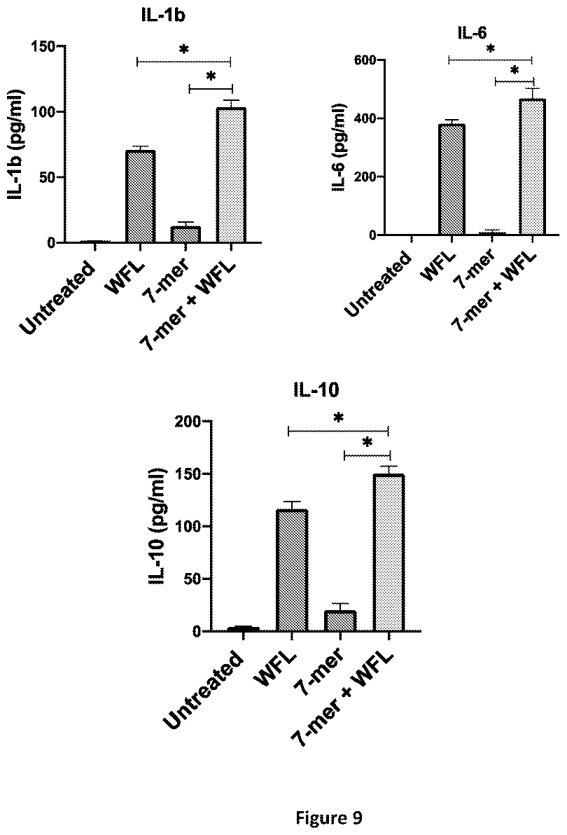

In one of the embodiments the inventors treated the THP-1 cells with about 200 ng/ml WFL (worm full length HSP70) protein; about 100 μg/ml 7-mer (Hepta N-acetyl Chitoheptaose) and combination of same concentrations of WFL and 7-mer (FIG. 9). The results indicated that WFL and 7-mer act synergistically and it combinatorial effect is better than their individual effect on immunomodulation of THP-1 cells.

An embodiment of the present invention may provide a homolog of human HSP70 derived from nematode Setaria digitata for prophylactic, therapeutic, medicinal treatment of respiratory disorders. Another embodiment of the present invention may provide that the homolog of human HSP70 derived from nematode Setaria digitata for prophylactic, therapeutic, medicinal treatment of respiratory disorders has 70-80% homology with the human HSP70.

The present invention has been described with reference to the exemplary embodiments and the drawings, but the present invention is not limited thereto. The described exemplary embodiments may be changed or modified by those skilled in the art to which the present invention pertains within the technical spirit of the present invention and within the scope equivalent to the appended claims. Further the invention should not be limited to the description of the embodiments therein. It will be obvious to those skilled in the art to make various changes, modifications and alterations to the invention described herein, to the extent that these changes, modifications and alterations do not depart from the scope of the invention; they are intended to be encompassed therein.

1. A protein homolog of human HSP70 derived from nematode Setaria digitata which is capable of treating and/or preventing sepsis, multiple organ dysfunction syndrome (MODS) or septic shock.

2. The protein as claimed in claim 1 wherein the protein has 70-80% homology with the human HSP70.

3. The protein as claimed in claim 1, wherein the protein improves immunomodulation in post-septicaemia in mammals.

4. The protein as claimed in claim 1, wherein the protein immunomodulates by activation of TLR4 (Toll like receptor 4) and/or TLR2 receptors.

5. A chito-oligosaccharide which is capable of treating and/or preventing sepsis, multiple organ dysfunction syndrome (MODS) or septic shock wherein the chito-oligosaccharide is selected from a group comprising of a derivative of hexa-N-acetyl chitohexaose, hepta-N-acetyl chitoheptaose or octa-N-acetyl chitooctaose.

6. The chito-oligosaccharide as claimed in claim 5, wherein the chito-oligosaccharide is hepta-N-acetyl chitoheptaose.

7. The chito-oligosaccharide as claimed in claim 5, wherein the chito-oligosaccharide immunomodulates by activation of TLR4 (Toll like receptor 4) and/or TLR2 receptors.

8. The chito-oligosaccharide as claimed in claim 5, wherein the chito-oligosaccharide is administered before and/or after onset of symptoms of sepsis.

9. A pharmaceutical composition comprising physiologically effective amounts of protein homolog of human HSP70 derived from nematode Setaria digitata and chito-oligosaccharide wherein the oligosaccharide is selected from the group comprising of a derivative of hexa-N-acetyl chitohexaose, hepta-N-acetyl chitoheptaose or octa-N-acetyl chitooctaose which is capable of treating and/or preventing sepsis, multiple organ dysfunction syndrome (MODS) or septic shock.

10. The pharmaceutical composition as claimed in claim 9, wherein the protein has 70-80% homology with the human HSP70.

11. The pharmaceutical composition as claimed in claim 9, wherein the protein immuno-modulates survival through post-septicaemia in a mammal.

12. The pharmaceutical composition as claimed in claim 9, wherein the composition immuno-modulates by activation of TLR4 (Toll like receptor 4) and/or TLR2 receptors.

13. The pharmaceutical composition as claimed in claim 12, wherein said sepsis is mild sepsis, severe sepsis, infection symptoms or sepsis caused by burn, acute laryngopharyngitis, ulcerative colitis, IBS (Irritable Bowel syndrome), rheumatic arthritis, degenerative arthritis, acute hepatitis, or chronic hepatitis.

14. The pharmaceutical composition comprising the protein and/or the chito-oligosaccharide as claimed in claim 9, further comprising of, additives, binders and excipients or a combination thereof.

15. A method of treatment or prevention of sepsis, MODS or septic shock in a mammal including human comprising administering an effective amount of protein homolog of human HSP70 derived from nematode Setaria digitata or chito-oligosaccharides wherein the chito-oligosaccharides is selected from the group comprising of a derivative of hexa-N-acetyl chitohexaose, hepta-N-acetyl chitoheptaose or octa-N-acetyl chitooctaose or a combination thereof together with a pharmaceutically acceptable carrier thereof to said mammal.

16. The method as claimed in claim 15, wherein the wherein the protein has 70-80% homology with the human HSP70.

17. The method as claimed in claim 15, wherein the chito-oligosaccharide is hepta-N-acetyl chitoheptaose.

18. A synthetic or recombinant nucleic acid encoding a protein homolog of human HSP70 derived from nematode Setaria digitate which is capable of treating and/or preventing sepsis, multiple organ dysfunction syndrome (MODS) or septic shock, or a chito—oligosaccharide, derivative, recombinant or part thereof as claimed in claim 5.

19. The nucleic acid as claimed in claim 18, wherein the nucleic acid encodes a protein having 70-80% homology with the human HSP70.

20. (canceled)

21. (canceled)
